(19) United States
(12) Patent Application Publication
(10) Pub. No.: US 2026/0354960 A1
(43) Pub. Date: Nov. 12, 2026

(54) PROSTHETIC DEVICE FOR TISSUE REGENERATION

(71) Applicants: UNIVERSITY OF CONNECTICUT, Farmington, CT (US); CONNECTICUT CHILDREN'S MEDICAL CENTER, Hartford, CT (US)

(72) Inventors: Christine Finck, South Glastonbury, CT (US); Heather Wanczyk, Tolland, CT (US); Joanne Walker, Burlington, CT (US)

(21) Appl. No.: 19/125,003

(22) PCT Filed: Oct. 27, 2023
(86) PCT No.: PCT/US2023/078063

§ 371 Date: Apr. 28, 2025

Related U.S. Application Data
(60) Provisional application No. 63/381,303, filed on Oct. 28, 2022.

(51) Int. Cl. A61F 2/04; A61L 27/26; A61L 27/36; A61L 27/38; A61L 27/54; A61L 27/56

(52) U.S. Cl. CPC A61F 2/04; A61L 27/26; A61L 27/3679; A61L 27/3813; A61L 27/3826; A61L 27/383; A61L 27/3834; A61L 27/54; A61L 27/56; A61F 2002/044; A61F 2210/0004; A61F 2240/002; A61L 2300/64; A61L 2430/22

(57) ABSTRACT

A prosthetic device for repairing a damaged organ, includes a porous scaffold adapted for hosting a population of therapeutic cells, the porous scaffold having an outer surface and an inner surface separated by a thickness. A median pore size in the porous scaffold is less than a limiting median dimension for the population of therapeutic cells; and, a population of therapeutic cells is seeded upon the scaffold. The therapeutic cells include autologous cells in an amount of between about 10,000 cells to about 15,000 cells per square millimeter of surface area of the porous scaffold. Methods of imaging, fabrication and use are provided.

### CROSS-REFERENCE TO RELATED APPLICATIONS

The present disclosure claims priority to, and the benefit of, U.S. Provisional Application No. 63/381,303, filed 28 Oct. 2022, which is incorporated by reference herein in its entirety for all purposes.

### BACKGROUND

### 1. Field of Invention

This disclosure relates to a prosthetic device for tissue regeneration. More specifically, this disclosure relates to a personalized prosthetic device that uses autologous cells for tissue regeneration.

### 2. Description of the Related Art

A variety of congenital esophageal abnormalities (malformations in the esophagus present at birth) occur in the population. Common types include esophageal atresia, tracheoesophageal fistula, and congenital esophageal stenosis. Esophageal atresia is the most frequent, occurring in approximately 1 in 2,500 to 4,500 live births. This condition involves an interruption of the esophagus, leading to an opening between its upper and lower segments. Long gap esophageal atresia is a subtype where the distance between the two segments exceeds 4 cm.

Surgical repair is the primary treatment for these conditions. In the case of esophageal atresia, surgeons often employ end-to-end anastomosis, where they join the two ends of the esophagus. Advantages of this approach include immediate restoration of esophageal continuity and generally lower complication rates. Disadvantages include the requirement for timely diagnosis and a limited applicability for long gap esophageal atresia.

For long gap esophageal atresia, alternative therapies such as the Foker technique or esophageal lengthening exist. These therapies involve staged procedures to gradually bring the two esophageal ends closer together. The advantages of these techniques include their applicability to more severe cases. The disadvantages encompass higher risk of complications and longer hospital stays.

Some techniques employ autologous donor tissues (i.e., intestine, colon, or small intestine) to bridge the gap. Unfortunately, since these are not functionally relevant tissues, significant long-term morbidities, such as Gastroesophageal Reflux Disease (GERD) can persist into adulthood. As such, it has been advocated that use of native esophageal tissue is best whenever possible.

Thus, what are needed are novel approaches that bridge a primary long gap, repair a failed esophageal atresia (EA) treatment, or repair segmental lesions of a hollow organ due to injury or disease, while simultaneously preserving the native tissue (e.g., esophageal tissue) are highly desired.

### SUMMARY OF THE INVENTION

In one embodiment, a prosthetic device for repairing a damaged organ is provided. The prosthetic device includes: a porous scaffold adapted for hosting a population of therapeutic cells, the porous scaffold including an outer surface and an inner surface separated by a thickness; and wherein a median pore size in the porous scaffold is less than a limiting median dimension for the population of therapeutic cells.

Therapeutic cells may be autologous cells, and the autologous cells may be stem cell, and the stem cells may be esophageal epithelial cells, adipose derived mesenchymal stromal cells, muscle cells, neural progenitor cells, induced pluripotent stem cells, bone marrow stem cells or a combination thereof. The porous scaffold may include a bioresorbable polymer. The porous scaffold may include a polyolefin, a fluoropolymer, a polysiloxane, a polyurethane, a polyester, a polyimide, a polyetherimide, or a combination thereof. The porous scaffold may include a polypropylene copolymer and a carbohydrate filler. The therapeutic cells may be present in an amount of about 10,000 cells to about 15,000 cells per square millimeter of surface area of the porous scaffold. The therapeutic cells may be disposed on the porous scaffold by seeding them into the porous scaffold.

In another embodiment, a method of repairing a damaged organ in a living being is provided. The method includes modeling the damaged organ from images taken from the living being; receiving a prosthetic device configured for repairing the damaged organ, the prosthetic device including: a porous scaffold adapted for hosting a population of therapeutic cells, the porous scaffold including an outer surface and an inner surface separated by a thickness; and wherein a median pore size in the porous scaffold is less than a limiting median dimension for the population of therapeutic cells; seeding the prosthetic device with therapeutic cells; and implanting the prosthetic device in the living being.

In another embodiment, a method of fabricating a prosthetic device adapted for repairing a damaged organ in a living being is provided. The method includes receiving a model of the damaged organ, the model developed from images taken from the living being; fabricating the prosthetic device by building a porous scaffold adapted for hosting a population of therapeutic cells, the porous scaffold including an outer surface and an inner surface separated by a thickness; and wherein a median pore size in the porous scaffold is less than a limiting median dimension for the population of therapeutic cells.

The fabricating may include additive manufacturing of the porous scaffold by fused deposition modeling where a fused deposition model includes instructions for depositing material layer-by-layer to create a three-dimensional object. The method may further include seeding the porous scaffold with therapeutic cells by bioprinting. The method may further include seeding the porous scaffold with therapeutic cells while rotating the porous scaffold. The images may be collected using computed tomography, magnetic resonance imaging, positron emission tomography, single-photon emission computerized tomography, ultrasound or a combination thereof. The method may further include inducing a paracrine effect in the autologous cells, the paracrine effect facilitating new tissue growth across a span of the scaffold; where the new tissue develops an original tissue of the organ, and the autologous cells may be stem cells, and the stem cells may be esophageal epithelial cells, adipose derived mesenchymal stromal cells, muscle cells, neural progenitor cells, induced pluripotent stem cells, bone marrow stem cells or a combination thereof.

In another embodiment, a prosthetic device for repairing a damaged organ is provided. The prosthetic device includes: a porous scaffold adapted for hosting a population of therapeutic cells, the porous scaffold including an outer surface and an inner surface separated by a thickness; and wherein a median pore size in the porous scaffold is less than a limiting median dimension for the population of therapeutic cells; and, a population of therapeutic cells seeded upon the scaffold, the therapeutic cells including autologous cells in an amount of between about 10,000 cells to about 15,000 cells per square millimeter of surface area of the porous scaffold. The damaged organ may be an esophagus.

### BRIEF DESCRIPTION OF THE DRAWINGS

FIG. 1A depicts a side view of an exemplary prosthetic device;

FIG. 1B depicts a cross-sectional view of the exemplary prosthetic device taken at AA′ of the FIG. 1A;

FIG. 2 is a view depicting exemplary regions of the scaffold that may have different levels of porosity from other regions of the scaffold;

FIG. 3A is a photomicrograph that depicts a scaffold manufactured using a polypropylene copolymer that contains dispersed carbohydrate particles, after the carbohydrate particles are removed;

FIG. 3B is a photomicrograph that depicts the scaffold of the FIG. 3A after it is seeded with therapeutic cells;

FIG. 4 is a depiction of the insertion of the prosthetic device depicted in the FIG. 1A in a tubular hollow organ; and

FIG. 5 is a flow chart that depicts a method for developing a personal prosthetic device for a patient.

### DETAILED DESCRIPTION OF THE INVENTION

Disclosed herein is a prosthetic device that may be used as an implant for development of new tissue or tissue regeneration on the inside or on the outside of a body of a living being. In an embodiment, it is preferably used on the inside of a body of a living being. The prosthetic device comprises a porous scaffold into which is disposed a plurality of therapeutic cells. The pores in the scaffold facilitate the anchoring of the therapeutic cells during the formation of new tissue when the prosthetic device is installed in or on the body of a living being. These therapeutic cells are disposed in the pores of the scaffold in an amount effective to form a cellular layer on the scaffold prior to being disposed in the body of a living being.

The prosthetic device is preferably used to facilitate initial generation or rebuilding of damaged or removed tissue in a gap (hereinafter “opening”) in a hollow internal organ such as for example, the esophagus, intestines, arteries, and the like. The opening may be only a small hole in the hollow organ, which needs a patch to facilitate reformation of damaged or removed tissue. In this event, the prosthetic device may be fashioned as a patch to plug the opening. On the other hand, the opening may be a tubular section configured to mimic the missing portion of hollow organ, in which case, the prosthetic device is used as a tubular bridge to connect opposing open ends of the hollow organ. The scaffold facilitates the formation of neo-tissue spanning the entire length of the implant, restoring continuity of the host tissue of the hollow organ (e.g., esophagus) or organs. The scaffold therefore serves as a supportive structure that mimics the extracellular matrix, providing a framework for the cells to attach, proliferate, and differentiate. The seeded scaffold is used in tissue engineering applications to regenerate or repair damaged tissues or organs.

In an embodiment, the therapeutic cells are autologous cells that are removed from the patient, stored and then seeded into the scaffold to facilitate tissue regeneration. As noted above, the scaffold may generally act as a bridge (in a hollow organ) to get fibrous tissue to grow over it in a circumferential manner. After the scaffold is removed or resorbed, the residual paracrine effects of the autologous cells produce new tissue that closely replicates the original tissue of the damaged organ. The autologous cells may be obtained via a fat pad biopsy from a patient. In an embodiment, adipose derived mesenchymal stem cells (Ad-MSCs) obtained from the fat pad biopsy are cultured in a controlled environment to promote their growth and expansion and used to seed the scaffold. This method of tissue regeneration results in the formation of tissue that closely resembles the original tissue of the patient and avoids some conventional problems associated with organ transplants such as organ rejection.

Disclosed herein too are methods for providing the prosthetic device in the form of a personalized therapeutic medical device (hereinafter “personalized prosthetic device”). The method comprises performing diagnostic imaging one or more organs in the body of a living being (the patient) prior to or after removal of a portion of the organ. Diagnostic imaging includes images obtained via a computed axial tomography (CAT) scan (now commonly referred to as a computed tomography (CT) scan), a magnetic resonance imaging (MRI) scan, a positron emission tomography (PET) scan, a single-photon emission computerized tomography (SPECT) scan, ultrasound, or a combination thereof. The digitized image may be converted to a computer aided drawing (CAD) using a design program, which can then be used to develop a personalized prosthetic device for the patient. The personalized prosthetic device may be manufactured using additive manufacturing and will have characteristics (e.g., structural features, physiological features, biological features) that are unique to the patient.

Additive manufacturing includes three-dimensional (3D) printing. Examples of 3D printing include fused deposition modeling (FDM) or 3D bioprinting. Additive manufacturing may be used to manufacture both the scaffold as well as the layer of therapeutic cells disposed thereon. The therapeutic cells that are initially deposited on the scaffold are preferably ones that stimulate and encourage tissue repair. It is therefore desirable to have both the materials that are used in the scaffold as well as those used as therapeutic cells to be available in the form of a printable ink that can be discharged from a nozzle to manufacture a customized prosthetic device.

In order to provide some context for the teachings herein, some aspects and terminology are now introduced.

Generally, as discussed herein, “fused deposition modeling (FDM),” which may also be referred to as “fused filament fabrication (FFF),” calls for extruding filaments of a resorbable non-toxic material, such as, for example, PLA (polylactic acid) or polyhydroxyalkanoates (PHAs), through a heated nozzle, melting the material and applying the material layer by layer to build a scaffold. Each layer of the scaffold is laid down one at a time until the part (e.g., the scaffold) is complete. Three dimensional (3D) bioprinting (also referred to herein as additive manufacturing) makes use of such techniques and further combine cells, growth factors, and/or biomaterials to fabricate biomedical parts.

As discussed herein, the term “therapeutic cells” generally refers to biological cellular materials useful for initiating therapy for a patient. Examples include autologous cells or autologous adipose derived mesenchymal stromal cells (Ad-MSC) as well as others. Ad-MSCs possess a high regenerative potential and immunomodulatory activities. Ad-MSCs have great potential in stem cell-based therapies and wound healing as well as in orthopedic, cardiovascular, or autoimmune diseases. Generally, mesenchymal stem cells (MSCs) are multipotent stem cells that can differentiate into a variety of cell types, including bone cells (osteoblasts), cartilage cells (chondrocytes), muscle cells (myocytes) and fat cells that give rise to marrow adipose tissue (adipocytes). Bioprinting may include therapeutic cells along with other materials, such as a carrier, to enable and/or nourish the therapeutic cells.

The term “scaffold” as used herein, may be interchangeable with similar terms such as “implant”, “stent,” “substrate” and, in some cases “prosthesis.” In some embodiments, the scaffold is tubular and suited for therapy of an opening (in normally occurring tissue inside the body of a living being) or other anomaly, such as a perforation.

The scaffold may be partially tubular, C-shaped, a stripe or of another shape such as an irregular or contoured shape. In short, the scaffold may exhibit any geometry deemed suitable by a medical practitioner in order to repair a damaged section of an organ present in the body of a living being. In some embodiments, the scaffold is configured for developing new or original tissue that was omitted, as is the case with long gap esophageal atresia (LGEA).

The term “living being” as used herein applies to any species of multicellular organism that has a capacity for one or more of homeostasis, organization, metabolism, growth, adaptation, response to stimuli, and reproduction. The term “living being” encompasses vertebrates and invertebrates. It encompasses mammals, fish, birds, reptiles and amphibians.

The term “autologous cells” refer to cells that are obtained from and used in the same individual. In other words, they are cells that are harvested from a person's own body for therapeutic or medical purposes, often for the purpose of transplantation or regenerative therapies. Autologous cells are advantageous in that they reduce the risk of rejection or immune reactions, as the cells are recognized as “self” by the immune system. Autologous stem cell transplantation (also called autogenous, autogenic, or autogenic stem cell transplantation) includes transplantation in which stem cells (undifferentiated cells from which other cell types develop) are removed from a person, stored, and later given back to that same person.

The paracrine effect refers to the phenomenon in which a cell secretes signaling molecules that act on nearby cells in the immediate microenvironment. These signaling molecules, which can include various growth factors, cytokines, and chemokines, influence the behavior and function of neighboring cells without directly affecting the cell that produced them. In the context of stem cell therapy, transplanted stem cells can exert a paracrine effect by releasing factors that promote tissue repair, modulate immune responses, and stimulate endogenous regenerative processes. It is involved in the coordination of cellular activities within tissues, helping to maintain tissue homeostasis and regulate physiological functions.

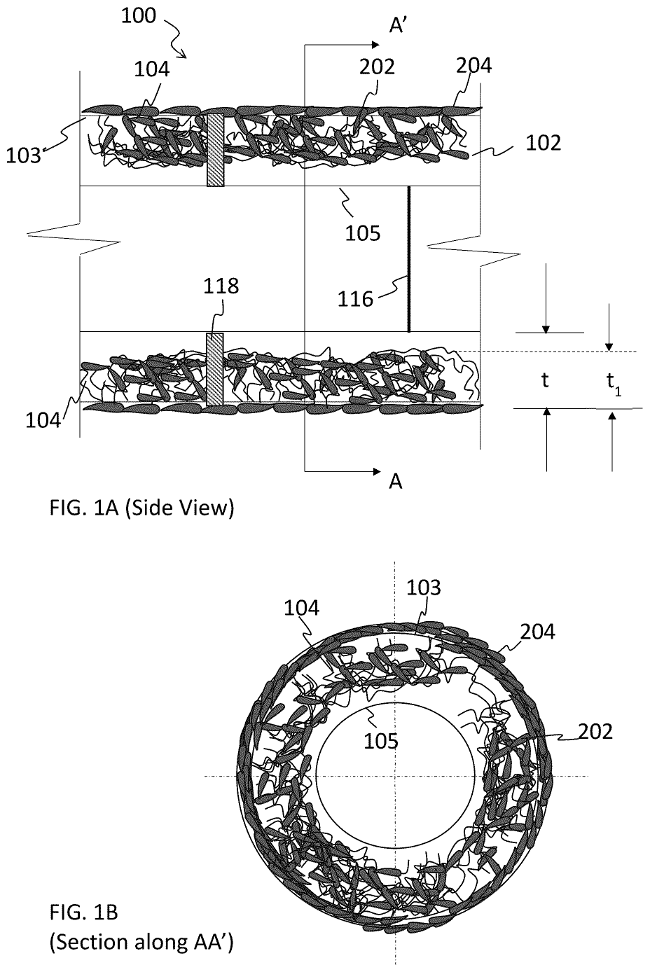

FIGS. 1A and 1B are depictions of a section of an exemplary prosthetic device 100 an implant for tissue regeneration on the inside or on the outside of a body of a living being. FIG. 1A depicts a side view of a section of the exemplary prosthetic device 100, while FIG. 1B depicts a cross-sectional view taken along section AA′ of the FIG. 1A. The prosthetic device 100 comprises a scaffold 102 into which and upon which are disposed a plurality of therapeutic cells 202 and 204 that facilitate tissue regeneration to replace damaged and/or removed tissue from the body of a living being. The scaffold 102 has an inner surface 105 and an outer surface 103 separated by a thickness “t” and between which lie a plurality of pores 104. The pores are in communication with the outer surface 103 but do not extend to contact the inner surface 105. As may be seen in the FIG. 1A, the pores extend to a depth of thickness t₁ from the outer surface 103. The ratio of t₁ to t depends upon a variety of factors such as the nutrients that are supplied to the prosthetic device, the location of the device in the body and the function and properties of the organ that is being rebuilt.

The plurality of pores 104 are filled with therapeutic cells 202. The therapeutic cells are seeded onto the scaffold in an amount effective to form a cellular layer 204 on the outer surface 103 of the scaffold. Seeding as used herein may include dripping the cells onto the scaffold, bioprinting the cells onto the scaffold, or a combination thereof. This will be discussed later. The cells that are seeded stay in position in the scaffold because of frictional forces between the cells and the pores in the scaffold. Other factors such as the use of decellularized tissues can also encourage adhesion.

In an embodiment, the scaffold 102 comprises a polymer that is biocompatible and is not rejected by the body. The polymer is preferably one that can be mixed with a suitable non-toxic, environmentally friendly solvent to form an ink that can be injected from a nozzle to form the scaffold via an additive manufacturing process. It is desirable for the polymer used in the scaffold to have a tear strength that can withstand suturing or stapling, since the prosthetic device will be bonded to existing tissue (in the body of the living being) during the process of tissue replacement.

The scaffold 102 is a porous body and may comprise a foam that has a porosity of 20 to 80 volume percent, preferably 30 to 60 volume percent, based on a volume of the entire scaffold 102. The porosity in the foam may be uniform or alternatively, can be tailored to be different in different regions depending upon the reinforcement desired in different portions of the tissue that is to be replaced. The term “foam” as used herein, is generally with reference to a manifestation of materials that exhibit a degree of porosity. The foam may be fabricated in any one or more of a number of ways, and therefore is not to be construed as limiting of the technology disclosed herein.

In an embodiment, the foam is preferably an open-cell foam and extends from the outer surface 103 of the scaffold to a depth of thickness t₁. In one embodiment, the inner surface 105 of the scaffold is a solid surface that is devoid of pores and is not in any fluid communication with the pores near the outer surface of the scaffold. In another embodiment, the pores can reach both surfaces—the outer surface and the inner surface. However, the cells never form a cellular layer on an inner surface of the scaffold. This because the average cell size is larger than the average pore size, which prevents the cells from diffusing to and growing on the inner surface of the scaffold. The porosity in the scaffold may be developed by virtue of the manufacturing process used (e.g., extruding struts randomly in all directions one atop the other to form a 3-dimensional porous network; this is accomplished via additive manufacturing), by using a blowing agent in the polymer (casting a polymer with a blowing agent in it and activating the blowing agent to form the foam) or by dispersing a soluble phase in a polymer and extracting the soluble phase by immersing the polymer in a suitable solvent. These manufacturing process for the scaffold and the prosthetic will be detailed later.

While FIGS. 1A and 1B depict the scaffold as being tubular with curved inner and outer surfaces that are parallel to each other, this does not always have to be the case. The outer surface 103 and inner surface 105 may be flat and/or curved depending upon the tissue that needs to be replaced or repaired. In an embodiment, the outer surface 103 is flat, while the inner surface 105 is curved or vice versa. In another embodiment, the inner surface 105 is parallel to the outer surface 103 and both surfaces are flat. In another embodiment, the inner surface 105 is parallel to the outer surface 103 and both surfaces are curved.

In an embodiment, the prosthetic device 100 has an outer surface 103 and an inner surface 105 that are concentric with one another (i.e., the cross-section area as seen in the FIG. 1B is a ring). In this event, the prosthetic device will be tubular in shape and can be inserted into a hollow organ to be used as an implant for tissue regeneration. In an embodiment, the cross-sectional area as seen in the FIG. 1B may be a partial ring. In other words, the prosthetic device is a partial tube that can be used as an implant for tissue regeneration.

In another embodiment, the prosthetic device can be a conduit (with concentric inner and outer surfaces) but can have an outer surface that is in the shape of a truncated cone. The diameter at one end of the prosthetic device is greater than the diameter at an opposing end of the device. Both ends of the truncated cone are open and can be attached to opposing ends of hollow organ to facilitate tissue regeneration.

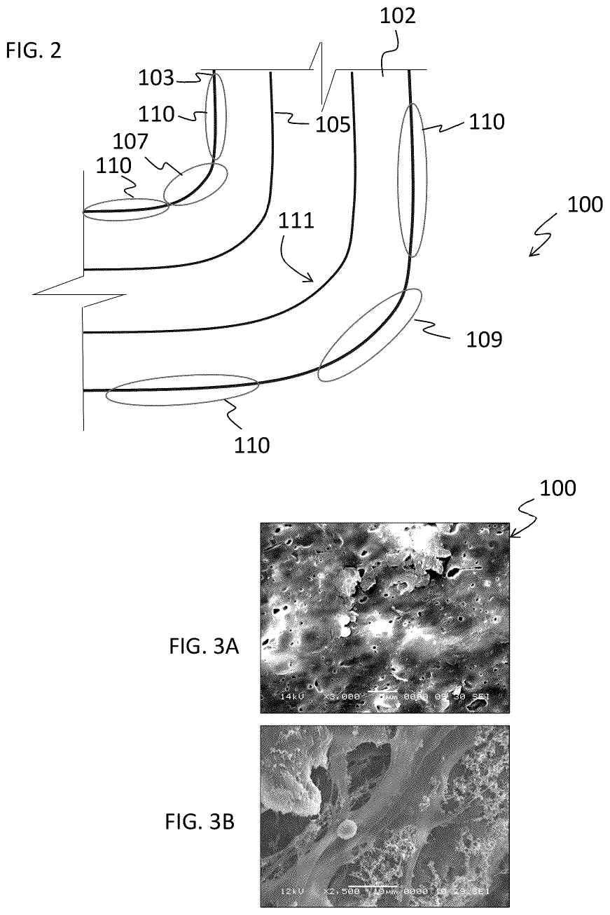

FIG. 2 depicts a scaffold 102 with a bent portion 111 (an elbow) that simulates a portion of the small or large intestine. Since those portions of the eventual repaired tissue that is located at the elbow 111 (region 107 at the inner bend and region 109 at the outer bend) will be subjected to a greater amount of flexing, it may be desirable to have a larger amount of tissue reinforcement at the elbow 111 when compared with the portions of the scaffold that do not have a bend such as regions 110. The increased tissue thickness at the inner and outer bend regions 107 and 109 can be generated by having a greater porosity in the bend regions when compared with those regions 110 that are not bent. The increased porosity will allow for the ingress of larger amounts of therapeutic cells (as well as hormones and other factors as determined by a paracrine effect), which will eventually result in a greater tissue thickness in the bend regions as compared with the regions that are not bent.

In another embodiment, the pore sizes in certain portions of the scaffold may be different from those in other regions of the scaffold. Pore size can be varied to change the rate and amount of therapeutic cell ingress and egress into and out of the scaffold. By varying the concentration of therapeutic cells in a particular region of the scaffold, the thickness of the tissue generated in that particular region may be varied.

In an embodiment, the scaffold may include pores having an average pore size of about 2 to about 50 μm, preferably 5 to 30 micrometers, and more preferably 10 to 25 micrometers. In an embodiment, the scaffold has a co-continuous microporous morphology. In an embodiment, the pores in the scaffold may have an average pore size of about 2 to about 5 μm, about 2 to about 4 μm, about 2 to about 3 μm, about 3 to about 6 μm, about 3 to about 5 μm, about 3 to about 4 μm, about 4 to about 6 μm, about 4 to about 5 μm, or about 5 to about 6 μm. A co-continuous morphology is a non-equilibrium morphology that is generated during mixing of two polymers. As such, it is an unstable morphology, and it starts changing through filament break-up and retraction as soon as the polymer blend in molten form emanates from the mixer. The blend may however remain co-continuous, if it is frozen fast.

For example, in an embodiment, the scaffold may include a co-continuous microporous morphology, wherein at least about 30% of the pores may have an average pore size of about 2 to about 50 μm. In an embodiment, the scaffold may include a co-continuous microporous morphology wherein at least 30% of the pores may have an average pore size about 2 to about 5 μm, about 2 to about 4 μm, about 2 to about 3 μm, about 3 to about 6 μm, about 3 to about 5 μm, about 3 to about 4 μm, about 4 to about 6 μm, about 4 to about 5 μm, or about 5 to about 6 μm. In another embodiment, for a scaffold having a microporous morphology at least about 35%, at least about 40%, at least about 45%, at least about 50%, at least about 55%, at least about 60%, at least about 65%, at least about 70%, at least about 75%, at least about 80%, at least about 75%, at least about 90%, at least about 95%, or at least about 98% of the pores may have an average pore size of about 2 to about 6 μm, about 2 to about 5 μm, about 2 to about 4 μm, about 2 to about 3 μm, about 3 to about 6 μm, about 3 to about 5 μm, about 3 to about 4 μm, about 4 to about 6 μm, about 4 to about 5 μm, or about 5 to about 6 μm. The microporous morphology or the co-continuous microporous morphology is desirable as it may enhance and/or promote cell adhesion to scaffold during the seeding process.

Generally, a median size of the pores in the scaffold is less than that of a median size of a limiting dimension of the therapeutic cells as may be disposed upon or seeded onto the scaffold. That is, the sizing of the microporous structures in comparison to the population of therapeutic cells is such that cell adhesion to scaffold during the seeding process as well as growth and therapy phases is enabled.

The polymers used to form the scaffold may be thermoplastic polymers, thermosetting polymers, blends of thermoplastic polymers, blends of thermosetting polymers, or a combination thereof. The polymer may also be a blend of polymers, copolymers, terpolymers, or a combination thereof. The polymer can also be an oligomer, a homopolymer, a copolymer, a block copolymer, an alternating copolymer, a random copolymer, a graft copolymer, a star block copolymer, a dendrimer, a polyelectrolyte (polymers that have some repeat groups that contain electrolytes), a polyampholyte (a polyelectrolyte having both cationic and anionic repeat groups), an ionomer, or the like, or a combination thereof. The polymers have number average molecular weights greater than 10,000 grams per mole, preferably greater than 20,000 g/mole and more preferably greater than 50,000 g/mole. The polymers have a number average molecular weight below 1,000,000 g/mole. The number average molecular weight may be measured using gel permeation chromatography (using a polystyrene standard).

In an embodiment, the polymers that form the scaffold are not bioresorbable and may be physically removed by a surgeon laparoscopically or endoscopically after the damaged tissue has been replaced. In another embodiment, the polymers that form the scaffold may be bioresorbable and are absorbed into the body after the damaged tissue is replaced.

Suitable polymers (that are not bioresorbable may be physically removed by a surgeon) used to form the scaffold include polyolefins (e.g., polyethylene, polypropylene, ethylene-a-olefin copolymers, ethylene propylene rubber (EPR), ethylene propylene diene monomer, or a combination thereof), fluoropolymers (e.g., polytetrafluoroethylene, fluorinated ethylene-propylene, polyethylenetetrafluoroethylene, perfluoroelastomers, vinylidene fluoride based copolymers, tetrafluoroethylene-propylene copolymers, perfluoropolyether, or a combination thereof) polysiloxanes, polyurethanes, polyesters, polyimides, polyetherimides, or a combination thereof.

Suitable bioresorbable polymers include polylactic-glycolic acid (PLGA), poly-caprolactone (PCL), copolymers of polylactic-glycolic acid and poly-caprolactone (PCL-PLGA copolymer), polyhydroxy-butyrate-valerate (PHBV), polyorthoester (POE), polyethylene oxide-butylene terephthalate (PEO-PBTP), poly-D, L-lactic acid-p-dioxanone-polyethylene glycol block copolymer (PLA-DX-PEG), or the like, or a combination thereof.

In an exemplary embodiment, the scaffold may include a thermoplastic elastomer (TPE) that includes a polypropylene copolymer (e.g., CAVERNA® PP), a soft polylactic acid (PLA) (e.g., soft-flexible PLA), a thermoplastic polyurethane (TPU), a plasticized copolyimide TPE (PCTPE), a thermoplastic co-polyester (TPC), or a mixture thereof.

As noted above, the scaffold 102 is a porous body (i.e., a foam) that may be manufactured by several different methods. In one embodiment, the scaffold 102 may be prepared using additive manufacturing. In additive manufacturing, the polymers listed above may be dissolved in a suitable solvent to form an ink that can be injected through a nozzle to form struts. By continuously extruding the struts to lie one atop another in random directions, a porous foam may be produced that can then be seeded with the therapeutic cells to produce the prosthetic device. After the formation of the foam, the solvent may be evaporated.

It is generally desirable to use solvents that are environmentally friendly and non-toxic. Volatile organic solvents may be used so long as the scaffold is substantially devoid of the solvent prior to its being used in the body of a living being. The evaporation of the solvent may be accomplished by drying the scaffold under a vacuum and/or at an elevated temperature (at a temperature greater than the boiling point of the solvent but below the flow temperature of the polymer). Suitable solvents for producing the ink are alcohols (e.g., methanol, ethanol, propanol, butanol, or the like, or a combination thereof), water, propylene carbonate, ethylene carbonate, butyrolactone, acetonitrile, benzonitrile, nitromethane, nitrobenzene, sulfolane, dimethylformamide, N-methylpyrrolidone, acetonitrile, nitromethane, benzene, toluene, methylene chloride, carbon tetrachloride, hexane, diethyl ether, tetrahydrofuran, or the like, or a combination thereof. Water and ethanol are preferred solvents.

In an embodiment, the polymer is used in the ink in an amount of 20 to 70 wt %, preferably 30 to 60 wt %, based on a total weight of the ink prior to being extruded through the nozzle during the additive manufacturing process to prepare the scaffold.

FIG. 3A is a photomicrograph that depicts a scaffold 100 manufactured via additive manufacturing using a polypropylene copolymer (e.g., CAVERNA® PP). The polypropylene copolymer is used in an additive manufacturing process where it is extruded from a nozzle at a temperature greater than its melting point to form the scaffold. The scaffold is then placed in a water bath for a period of time to dissolve a carbohydrate that is dispersed in the polypropylene copolymer. The removal of the carbohydrate particles leaves behind a microporous co-continuous network as seen in the FIG. 3A. FIG. 3B is a photomicrograph that depicts the microporous co-continuous network of the FIG. 3A after it is seeded with therapeutic cells.

In another embodiment, a polymer that is used to manufacture the scaffold is first dissolved in a solvent to form and. The ink is extruded from a nozzle to form struts. Each successive extruded strut is oriented in a different direction and is disposed on a preceding strut. The plurality of randomly oriented struts disposed one atop another form a porous network (i.e., a foam) upon drying. The foam upon drying may be used as a scaffold. The scaffold may then be seeded with therapeutic cells.

In another embodiment, the scaffold may be manufactured by mixing a blowing agent, a suitable solvent and a suitable polymer to form a blowing agent-polymer mixture, casting the polymer-blowing agent mixture into an optional mold and activating the blowing agent by raising the temperature of the polymer-blowing agent mixture. If a mold is not used, then the foam may be machined to the desired shape prior to seeding the foam with the therapeutic cells to form the prosthetic device.

In an embodiment, the foam may be first manufactured by mixing the polymer with a suitable blowing agent such that the polymer-blowing agent mixture exists in a single phase. The polymer-blowing agent mixture is retained at a pressure and temperature that is effective to retain the combination in a single phase until the combination is ready for use. When desired, the combination is pumped or poured into a mold and the pressure and/or temperature is changed to facilitate binodal decomposition which results in the nucleation and growth of cells in the polymer to form the foam. When the pressure and/or temperature in the cavities is changed, the blowing agent nucleates and grows in the polymer matrix resulting in the formation of a polymeric foam.

The blowing agent can be a physical blowing agent or a chemical blowing agent. Physical blowing agents typically undergo phase separation from the organic polymer (in which they are dissolved) upon experiencing a change in environmental conditions to produce the foam, while chemical blowing agents are those that undergo reaction or decomposition upon experiencing a change in environmental conditions to release a gas, which results in the formation of the foam.

Physical blowing agents include chlorofluorocarbons (CFCs), hydrochlorofluorocarbons (HCFCs), hydrocarbons (e.g., pentane, butane, isopentane, cyclopentane, or the like), and liquid or supercritical carbon dioxide. Chemical blowing agents include isocyanate and water for polyurethane, azodicarbonamide for vinyl, hydrazine or sodium bicarbonate for other thermoplastics.

In another embodiment, a thermally expandable microsphere can be used as the blowing agent. The microsphere is formed of a gas proof polymeric shell (e.g., polyacrylonitrile or polyvinylidene chloride) that encapsulates a (cyclo)aliphatic hydrocarbon (e.g., liquid isobutene). When the thermally expandable microspheres are subjected to temperatures of about 50 °C to about 200 °C, the polymeric shell softens and the (cyclo)aliphatic hydrocarbon expands, thereby promoting an increase in the volume of the microspheres. When expanded, the microspheres have a diameter 3.5 to 4 times their original diameter, as a consequence of which their expanded volume is about 50 to 60 times greater than their initial volume in the unexpanded state. An example of such thermally expandable microspheres are EXPANCEL® DU microspheres which are marketed by Nouryon Industries.

The blowing agent may be used in amounts of 5 to 30 wt %, preferably 8 to 20 wt %, based on a total weight of the scaffold prior to foaming and seeding with the therapeutic cells.

In yet another embodiment, the scaffold may be manufactured by extruding the polymer-blowing agent mixture through a nozzle in an additive manufacturing process. The polymer-blowing agent mixture may be extruded as struts atop one another in random directions (as detailed above with respect to FIG. 3A) to form a first foam. The blowing agents may then be activated by raising the temperature to form a second foam whose characteristics (foam density, cell shape, and the like) are different from the first foam. The second foam is then seeded with the therapeutic cells to form the prosthetic device.

In yet another embodiment, the polymers listed above (as scaffold materials) may be mixed with suitable soluble materials to form a composite. The composite is molded or cast (into a suitable mold) to form a desired shape, which is then subjected to solubilization in a suitable solvent to remove the soluble materials from the composite. The removal of the soluble materials leaves behind a porous scaffold that can be seeded with the therapeutic cells to form the prosthetic device.

Examples of soluble materials that may be extracted by solubilization include gelatin, gellan gum, carrageenan, guar gum, psyllium seed gum, yam starch powder, alginate, seaweed flour, tragacanth gum, karaya gum, curdlan, soy polysaccharides, alginic acid, carboxymethylcellulose, agar-agar, carrageenans, locust bean gum, gelatin, alginate, arabinoxylan, arrowroot, cassia gum, cellulose, gum Arabic, karaya gum, konjac, kuzu, maltodextrin, marshmallow root, pectin, sodium alginate, starch, xanthan gum, b-glucan or a combination thereof. The aforementioned soluble materials are soluble in water and/or alcohol. A preferred alcohol is ethanol.

The soluble materials used to facilitate porosity generation in the scaffold may be used in amounts of 40 to 80 wt %, preferably 50 to 70 wt %, based on a total weight of the scaffold prior to solubilization.

The soluble materials may be dissolved by disposing the scaffold in a solvent at temperature of 60 to 90 °C, preferably 75 to 85 °C for a period of 10 to 40 hours, preferably 25 to 35 hours. The removal of the soluble materials may create a microporous morphology or a co-continuous microporous morphology in the scaffold.

In an embodiment, the scaffold may include a thermoplastic elastomer (TPE) (e.g., a polypropylene copolymer) that has a water-soluble phase that dissolves (e.g., with treatment with water) to create a microporous morphology or a co-continuous microporous morphology (e.g., CAVERNA® PP). For example, the method may further include removing the water-soluble carbohydrate phase by incubating the scaffold including a thermoplastic elastomer (TPE) that has a water-soluble phase in an aqueous solution (e.g., water or distilled water).

Generally, CAVERNA® PP is a polypropylene copolymer filament with a carbohydrate filler. CAVERNA® PP has a water-soluble phase that dissolves to create a co-continuous microporous morphology. This material offers a unique microporous morphology and provides highly uniform pore size and distribution that result in good print fidelity. CAVERNA® PP is available from Infinite Material Solutions, LLC of River Falls, Wisconsin.

When the additive manufacturing of the scaffold is to be performed with a filament (e.g., a polypropylene filament, such as CAVERNA® PP) that has a water-soluble phase, it is placed in a solvent (e.g., water and/or alcohol) having a temperature of about 75 °C to about 85 °C for about 12 hours to about 36 hours, preferably about 18 hours to about 30 hours. For example, the solvent for treating the scaffold may have a temperature of about 75 °C to about 85 °C, about 75 °C to about 82 °C, about 75 °C to about 80 °C, about 78 °C to about 85 °C, or about 75 °C to about 82 °C (e.g., about 80 °C).

With reference now again to the FIG. 1A, the scaffold 102 may be provided with one or more predetermined fracture lines 116 across the inner surface 105 of the scaffold to facilitate an easy tearing off of the scaffold (and removal from the organ) after it has facilitated the formation of new tissue that bridges the span across the scaffold. This fracture line may be a perforation or a notch that is scored across the inner surface 105 of the scaffold and that promotes the scaffold to tear easily when it is being removed after it has served its purpose.

In another embodiment, when the scaffold 102 is manufactured predominantly from a polymer that is not resorbable, one or more small portions of the scaffold may be manufactured from a resorbable material 118. The one or more resorbable portions of the scaffold are generally uniformly spaced, such that the scaffold breaks up into a number of uniform small parts at about the time that the tissue bridging is completed.

When the prosthetic device 100 is inserted in the organ to facilitate tissue regeneration, these resorbable material 118 portions are gradually resorbed (absorbed) into the body of the living being. The rate of resorption is adjusted to be slower than the rate of tissue regeneration, such that the scaffold splits into a number of smaller sections (because the resorbable portions have been absorbed) that can be laparoscopically or endoscopically removed when the scaffold has served its purpose.

In an embodiment, the scaffold may optionally contain therapeutic and pharmaceutically biologically active agents to reduce pain, swelling, risk of infection, and other side effects from surgery. The pharmaceutically and biologically active agents include anti-analgesic agents, anti-arrhythmic agents, anti-bacterial agents, anti-cholinergic agents, anti-coagulant agents, anti-convulsant agents, anti-depressant agents, anti-diabetic agents, anti-diuretic agents, anti-fungal agents, anti-hypertensive agents, anti-inflammatory agents, anti-neoplastic agents, anti-nootropic agents, anti-retroviral agents, anti-tuberculosis agents, anti-tussive agents, anti-ulcerative agents, anti-viral agents, or the like, or a combination thereof.

The porous scaffold thus obtained may be seeded with therapeutic cells. In an embodiment, the therapeutic cells are autologous stem cells. Autologous stem cell transplantation (also called autogenous, autogenic, or autogenic stem cell transplantation) includes transplantation in which stem cells (undifferentiated cells from which other cell types develop) are removed from a person, stored, and later given back to that same person.

In an embodiment, the therapeutic cells used to seed the scaffold may include esophageal epithelial cells, adipose derived mesenchymal stromal cells (Ad-MSC), esophagus cells (e.g., decellularized esophagus), muscle cells (e.g., muscle cells, myocytes, and/or muscle cells derived from adipose tissue), neural progenitor cells (e.g., neural progenitor cells and/or neural progenitor cells derived from adipose tissue), or a combination thereof. For example, in an embodiment, the personalized implant may include (e.g., through seeding, such as described herein) pig esophagus cells (e.g., decellularized pig esophagus), muscle cells (e.g., muscle cells, myocytes, and/or muscle cells derived from pig adipose tissue), neural progenitor cells (e.g., neural progenitor cells and/or neural progenitor cells derived from pig adipose tissue), or a combination thereof.

The aforementioned autologous stem cells may be collected through a minimally invasive technique, such as a fat pad biopsy. This procedure is relatively simple and less invasive compared to other methods of obtaining stem cells. Once obtained, the desired tissue (such as, for example, the adipose tissue) is processed to isolate the desired stem cells (such as, for example, the Ad-MSCs), which are then cultured in a controlled environment to promote their growth and expansion.

After a sufficient number of cells have been cultured, they can be used to seed a scaffold. The scaffold is seeded by dripping a solution of the desired stem cells for a suitable period of time. The stem cells diffuse into the pores of the scaffold and form a layer on the surface of the scaffold as previously depicted and described with respect to the FIGS. 1A and 1B. In an embodiment, the seeding the scaffold with therapeutic cells may include disposing about 10000 cells to about 15000 cells per square millimeter of external surface area of the scaffold. In an embodiment, the seeding may include disposing about 10000 cells to about 11000 cells, about 11000 cells to about 12000 cells, about 12000 cells to about 13000 cells, about 13000 cells to about 14000 cells, about 14000 cells to about 15000 cells per square millimeter of surface area of the scaffold or more.

The scaffold serves as a supportive structure that mimics the extracellular matrix, providing a framework for the cells to attach, proliferate, and differentiate. The seeded scaffold can then be used in tissue engineering applications to regenerate or repair damaged tissues or organs. The extracellular matrix (ECM) is a complex three-dimensional network of extracellular macromolecules, such as collagen, elastin, proteoglycans, and multi-adhesive matrix proteins, that provide structural and biochemical support to the surrounding cells. When proteins or decellularized tissue are added to the surface of a scaffold, they can mimic the natural ECM environment, providing structural support and biochemical cues to the cells seeded on the scaffold. This can help promote cell attachment, proliferation, and differentiation, which are essential for tissue regeneration and engineering applications. The presence of these components can facilitate cell adhesion and the formation of new tissue, leading to improved integration and functionality of the implanted scaffold within the host tissue. Moreover, the addition of proteins or decellularized tissue can enhance the biocompatibility and bioactivity of the scaffold, making it more conducive to supporting cell growth and tissue development. It is particularly useful in the reconstruction of damaged hollow organs such as the esophagus, the intestines, large arteries, or the like. The scaffold seeded thus allows for the creation of tissue constructs that closely resemble native tissues, promoting better integration and functionality when implanted in an organ in the body of a living being. The scaffold facilitates the formation of new tissue that may extend across the entire length of the implant, restoring continuity of the host tissue of the hollow organ (e.g., esophagus) or organs. The scaffold therefore serves as a supportive structure, providing a framework for the cells to attach, proliferate, and differentiate. In an embodiment, the decellularized tissue is decellularized pig tissue.

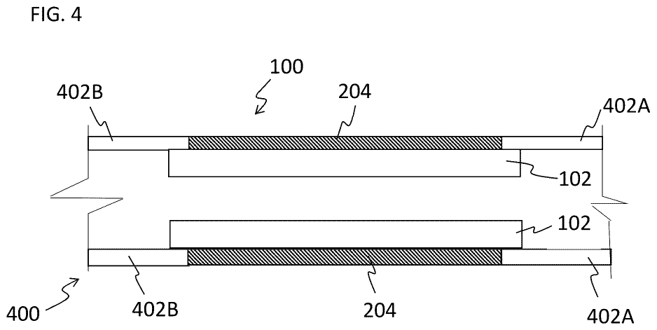

FIG. 4 is a depiction of the insertion of the prosthetic device 100 (previous depicted in the FIG. 1A) in a tubular hollow organ 400. The hollow tubular organ 400 has a portion between its opposing tubular ends 402A and 402B that is removed (because it was diseased or damaged). This portion of damaged tissue needs to be replaced to enable normal functioning of the hollow organ. This replacement may be accomplished using the prosthetic device 100 of the FIG. 1A. For purposes of simplicity, the prosthetic device of the FIG. 4 is depicted as only the scaffold 102 having a therapeutic cell layer 204 disposed thereon. The opposing ends of the scaffold 102 are inserted into the opposing tubular ends 400A and 400B of the hollow organ 400. The therapeutic cell layer 204 (where the therapeutic cells are appropriately chosen depending upon the organ being repaired) contacts the original tissue at the opposing tubular ends 402A and 402B of the hollow organ. In an embodiment, the therapeutic cell layer 204 may overlap with the original tissue at the opposing tubular ends 402A and 402B.

In an embodiment, the scaffold 102 may be sutured or stapled to original tissue located at the opposing tubular ends 402A and 402B of the hollow organ 400. In another embodiment, the therapeutic cell layer 204 may also be sutured, stapled or adhesively bonded to the original tissue located at the opposing tubular ends 402A and 402B of the hollow organ 400. During the period of time that the scaffold is located in the hollow organ 400, new tissue begins to form across the span of the scaffold. Paracrine effects of the therapeutic cells (used in the therapeutic layer 204 and in the pores of the scaffold (not shown here)) facilitate new tissue growth across the span of the scaffold that closely replicates the original tissue of the damaged hollow organ 400. The paracrine effect signals the release of hormones and other growth factors from neighboring cells that ensure that the tissue around the wound responds in a certain way to heal the wound. In an embodiment, the paracrine effect ensures that some of therapeutic cells contained in layer 204 act to stimulate the patient's cells to repair the diseased tissue, without these therapeutic cells contributing directly to the new tissue. This method of tissue regeneration results in the formation of tissue that closely resembles the original tissue of the patient. It also avoids problems traditionally associated with organ transplants such as organ rejection.

When it is determined that the bridging tissue formed across the span of the scaffold (from tubular end 402A to opposing tubular end 402B) is sufficiently strong and can function normally, the scaffold 102 may be removed laparoscopically or endoscopically. In an embodiment, the scaffold 102 may be gradually resorbed into the body and secreted with effluents normally secreted by the body. When the scaffold is gradually resorbed into the body, it is desirable that the rate of growth of new tissue exceeds the rate of resorption of the scaffold into the body. This will prevent the structure from coming apart before the organ is properly repaired and functioning normally.

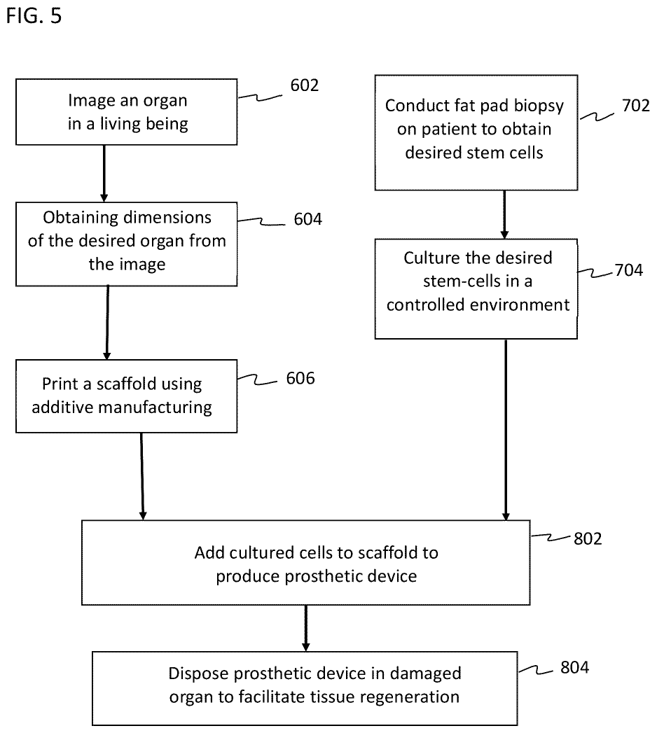

The method of creation of a personalized prosthetic device for providing individualized care will now be detailed. FIG. 5 depicts a method for manufacturing the prosthetic device for use on a particular living being. The method comprises imaging the damaged organ from the living being (602). As noted above, the imaging may include images obtained using computed tomography (CT) scans, magnetic resonance imaging (MRI) scans, a positron emission tomography (PET) scans, single-photon emission computerized tomography (SPECT) scans, ultrasound imaging, or a combination thereof. The data from these images may then be transferred to a microprocessor and analyzed to determine organ dimensions such as inner diameter, outer diameter, length of damaged region, length of organ to be removed, and the like (604). In an embodiment, one or more of the foregoing images of the patient may be converted or translated into a computer-aided design (CAD) file (e.g., stereolithography (STL) file) for additive manufacturing or bioprinting of the scaffold. For example, the method may further include modeling the scaffold in a computer-aided design (CAD) file (e.g., stereolithography (STL) file) for additive manufacturing or bioprinting the scaffold.

For example, the computerized tomography (CT) scan may be translated or converted into a computer-aided design (CAD) file (e.g., stereolithography (STL) file) of the scaffold utilizing, e.g., 3D Slicer® (Earth, Texas). Generally, 3D SLICER® is software for visualization, processing, segmentation, registration, and analysis of medical, biomedical, and other three-dimensional (3D) images. For example, the method may further include uploading the computer-aided design (CAD) file (e.g., stereolithography (STL) file) of the scaffold to a computer-aided design (CAD) program, e.g., SOLIDWORKS (Dassault Systèmes; Waltham, Massachusetts), for manufacturing. In an embodiment, manufacturing may include modifying the construct (e.g., modifying the construct of the computer-aided design file) file, developing three-dimensional (3D) printing programming language (e.g., B-programming language or G-code), or a combination thereof.

In an embodiment, a computer program product includes instructions which, when executed by a computer, causes the computer to carry out a method of preparing a personalized implant as described herein. For example, the computer program product includes instructions which, when executed by a computer, causes the computer to carry out: translating anatomical information from a computerized tomography (CT) scan of a recipient subject to produce a three-dimensional (3D) computer-aided design (CAD) of a scaffold for the recipient subject; and controls the manufacture of the scaffold via additive manufacturing or three-dimensional (3D) printing to produce a scaffold or a scaffold that includes a microporous morphology or a co-continuous microporous morphology.

The computer-aided design file may then be used to direct the motion of a nozzle of an additive manufacturing device to produce the scaffold (606). The desired polymer (in the form of an ink) is charged to the nozzle, which then undergoes directed motion to produce the porous scaffold. The scaffold will have dimensions that are relevant to the particular patient based on the images originally obtained.

In an embodiment, the additive manufacturing may be fused deposition modeling (FDM). FDM works by depositing material layer by layer to create a three-dimensional object.

In the FDM process, a thermoplastic filament is fed through a heated printer head, which then melts the material. The melted material is extruded through a fine nozzle onto a build platform, where it cools and solidifies, creating a thin layer of the object. The printer head moves along predetermined paths, depositing the material layer by layer according to the design specifications provided by the computer-aided design (CAD) program.

In another embodiment, the additive manufacturing may include bioprinting. Bioprinting is an additive manufacturing technique that involves the precise deposition of biological materials, such as cells, biomaterials, and bioactive factors, to create functional, living tissue-like structures. In bioprinting, specialized 3D printers are used to deposit bio-inks layer by layer in a controlled manner, following a digital design or blueprint (that may be provided by the computer-aided design (CAD) program). These bio-inks include living cells that are suspended in a biomaterial matrix, providing structural support and promoting cell attachment, growth, and differentiation. Various types of cells, including stem cells, primary cells, and cell lines, can be used in the bioprinting process, depending on the desired tissue or organ being fabricated.

In an embodiment, the bioprinting may include embedding autologous cells (e.g., esophageal epithelial cells, adipose derived mesenchymal stromal cells (Ad-MSC) in the porous scaffold.

Simultaneously with the imaging being conducted on the patient, a fat pad biopsy may be conducted on the patient to obtain stem cells (702). The desire stem cells are isolated from the biopsy and then cultured to obtain a sufficient quantity of these particular cells (704). The cultured cells are then dripped on the porous scaffold as it is rotated within a bioreactor to form the prosthetic device (802). This is termed seeding the scaffold. In an embodiment, seeding may include treating the scaffold with a substance to enhance cell adhesion. The substance may include an extracellular matrix component such as, for example, laminin, fibronectin, entactin, collagen,, decellularized extracellular matrix, or a mixture thereof. In an embodiment, the seeding may be conducted in a sterile bioreactor by adding the therapeutic cells to the scaffold.

The prosthetic device is then disposed into a hollow organ where tissue regeneration is desired (804). Several of these process-notably (702), (704), (802) and (804) from the FIG. 5 have been detailed above and will therefore not be described again in the interests of brevity.

In an embodiment, the process (802) may be repeated several times. In other words, the scaffold with the therapeutic cells seeded thereon may be incubated at a temperature of 35 °C. to 40 °C. for a period of time, followed by another seeding process where additional therapeutic cells are dripped onto the scaffold. The process of seeding followed by incubation may be repeated 2 to 6 times in order to increase the number of cells seeded per unit area of the scaffold surface. Each incubation may be conducted for 10 to 60 minutes.

In an embodiment, during the incubating, the scaffold with therapeutic cells may be exposed to a CO₂ concentration of about 4 to about 6% CO₂ (e.g., about 5% CO₂). The scaffold with the therapeutic cells disposed thereon may be placed in a sterile bioreactor and prevented from contacting any surface that could contaminate the scaffold. In an embodiment, during incubation, the scaffold with therapeutic cells disposed thereon may be in contact with growth media that facilitate growing the therapeutic cells. The growth media comprises neurotrophic and myotrophic growth factors, non-essential nutrients, glutamine, basal media, or a combination thereof.

In an embodiment, the scaffold with the therapeutic cells disposed thereon may be placed in a hermetically sealed package that provides (i) a sterile environment for the scaffold, (ii) an environment with the desired humidity for the scaffold, or (iii) a combination thereof. This enables the scaffold to be manufactured by a third party and to be stored in a warehouse for shipping as needed.

The method disclosed herein is advantageous because it may be used to produce a customized implant to repair a gap, a perforation or segmental lesion in a hollow organ or between hollow organs while preserving the native tissue of the hollow organ or organs. Such organs may include the esophagus, the small intestine, the large intestine, the colon, an artery, or the like. It may be used to repair damage caused by Type A esophageal atresia, Type B esophageal atresia, Type C esophageal atresia, Type D esophageal atresia, or long gap esophageal atresia (LGEA). It may also be used to repair damage caused by caustic burns to esophagus as well as adult conditions such as scleroderma.

The prosthetic device and the method of manufacturing and using it is exemplified by the following non-limiting examples. In the examples, the scaffold is used for esophageal and gastrointestinal tissue therapy and for specific conditions. However, it should be recognized this is illustrative only and that this technology may be useful for curing other pathologies.

### EXAMPLES

### Example 1: CAVERNA® PP Characterization

Three-dimensional (3D) scaffold test blocks for Examples 1-3 were printed via fused deposition modeling (FDM) with CAVERNA® PP, which is a polypropylene copolymer filament that has a water-soluble phase that dissolves in water to create a co-continuous microporous morphology. Scaffold test blocks (5 mm×5 mm) were dissolved in distilled water at 80 °C for 6 hours or 24 hours. The scaffold test blocks were examined via Scanning Electron Microscope (SEM) for the level of dissolution and to see if the desired pore size of about 3 to about 5 μm was obtained. The pores were not fully formed in the scaffold test blocks with the 6-hour incubation (now shown). As such, a 24-hour dissolution period was utilized for the rest of Examples 1-3. This 24-hour dissolution produced pore sizes of about 3 to about 5 μm. The microporosity was seen to be interconnected.

### Example 2: Cell Viability Examination

Autologous adipose derived mesenchymal stromal cells (Ad-MSCs) were derived from a patient with tracheoesophageal fistula (TEF) and suspended in StemXVivo® Mesenchymal Stem Cell Expansion Media (R&D Systems®, Minneapolis, MN, USA). The 3D printed scaffold 8 mm punches were placed in a well of a Corning® Costar® TC-Treated 12-Well Plate (Corning, NY, USA). Fifty microliters of cells (450,000 cells) were placed on the scaffold test blocks and incubated at 37 °C for 30 minutes to permit adherence of the cells to the scaffold. One mL of StemXVivo® Mesenchymal Stem Cell Expansion Media was added to the well and incubated for 24 hours at 37 °C-5% CO_{2.}

After the 24-hour incubation, the cells were examined for cell viability via LIVE/DEAD Cell Viability/Cytotoxicity Kit (ThermoFisher SCIENTIFIC Cat. No. L3224; hereinafter, “Live/Dead Assay”; Waltham, MA, USA) wherein a cell stained with Calcein-AM (green) denotes a live cell and a cell stained with ethidium bromide (red) denotes a dead cell. Fluorescent images of the Live/Dead Assay of scaffold test blocks were examined. All cells were viable within the well, adjacent to the 3D printed scaffold. Thus, no toxicity was observed by direct contact with the 3D printed scaffold or via leaching components from the same. This demonstrates that the 3D printed material was not toxic to the cells within a 24-hour incubation.

### Example 3: Cell Seeding Examination

Each 3D printed scaffold test block (5 mm×5 mm) was incubated in 40% ethanol for 15 minutes in a well of a Corning® Costar® TC-Treated 12-Well Plate. The ethanol treated scaffolds were rinsed twice with phosphate buffered saline, and then dried for 30 minutes in an incubator at 37 °C-5% CO₂. About 38,000 esophageal epithelial cells in 30 μL of Keratinocyte growth media were seeded on the 3D printed scaffold test blocks. After an hour incubation at 37 °C-5% CO₂, 1 mL of media was added to each well and incubated at 37 °C-5% CO₂ for an additional 24 hours.

After 24 hours of incubation, the cell seeded scaffold test blocks were examined for cell viability with the Live/Dead Assay. Exemplary fluorescent images, at increasing magnification, of the Live/Dead Assay of 3D printed scaffold text blocks were obtained demonstrating cell adhesion and viability of the seeded cells after a 24-hour incubation at 37 °C-5% CO₂. The images also demonstrated that the cells are proliferating on the surface of the 3D printed scaffold.

The cell seeded scaffolds were further incubated 144 hours (6 days) at 37 °C-5% CO₂. After the 6 days of incubation, the scaffold test blocks seeded with esophageal epithelial cells were examined by SEM. All images showed that the scaffold test blocks were viable.

### Example 4: Scaffold Characterization

3D printing was utilized to create a scaffold (12 mm diameter×50 mm length×0.5 mm thick) using Caverna® PP. SOLIDWORKS (Dassault Systèmes; Waltham, Massachusetts) was utilized to produce a computer-aided design (CAD) of the scaffold having a diameter of 12 mm diameter, a length of 50 mm, and a thickness of 0.5 mm. The resultant stereolithography (STL) file was printed on a PRUSA three-dimensional (3D) printer (Prusa Research a.s.; Prague, Czech Republic).

The scaffold was placed in a sterile bioreactor, and a total of 13 million cells (Ad-MSC) were seeded on the scaffold. The cells were suspended in 1.25 mL of StemXVivo® Mesenchymal Stem Cell Expansion Media and were seeded on the scaffold over the course of three rounds of seeding, each with a 25-minute incubation at 37 °C-5% CO₂. After seeding, approximately 350,000 cells were placed on the bottom of the bioreactor, and 250 μL of StemXVivo® Mesenchymal Stem Cell Expansion Media added to the bioreactor, submerging the scaffold in media.

After incubating the scaffold for about 2 hours with the seeded cells, a portion of the media was collected from the bioreactor and an automated hemacytometer cell counter used to count the number of cells in the media via trypan blue staining, thereby quantifying the number of cells that had not adhered to the scaffold. The media contained about 1,680,000 of the 13 million cells seeded on the tumular scaffold. Thus, about 86% of the seeded cells successfully adhered to the scaffold.

After 24 hours of incubation, five 8 mm punch biopsies were taken from the seeded scaffold and tested for cell viability using the Live/Dead Assay described herein.

Exemplary fluorescent images obtained from the Live/Dead Assay performed on the punch biopsies shows that the scaffolds were viable.

Coverage and cellular viability of adipose derived mesenchymal stromal cells (Ad-MSCs) seeded on scaffolds for 6 days was studied. Immunofluorescent images show the coverage and viability of adipose derived mesenchymal stromal cells (Ad-MSCs) seeded on three-dimensionally printed scaffolds coated with ethanol alone, fibronectin, or laminin. The three-dimensionally printed discs (10 mm) seeded with adipose derived mesenchymal stromal cells (Ad-MSCs) were stained with calcein and ethidium bromide to assess the viability after one and six days in culture. The data demonstrates that the scaffold is non-toxic and biocompatible.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

What is claimed is:

1. A prosthetic device for repairing a damaged organ, the prosthetic device comprising:
a porous scaffold adapted for hosting a population of therapeutic cells, the porous scaffold comprising an outer surface and an inner surface separated by a thickness; and
wherein a median pore size in the porous scaffold is less than a limiting median dimension for the population of therapeutic cells.

2. The prosthetic device of claim 1, where the therapeutic cells are autologous cells.

3. The prosthetic device of claim 2, where the autologous cells are stem cells.

4. The prosthetic device of claim 3, where the stem cells are esophageal epithelial cells, adipose derived mesenchymal stromal cells, muscle cells, neural progenitor cells, induced pluripotent stem cells, bone marrow stem cells or a combination thereof.

5. The prosthetic device of claim 1, where the porous scaffold comprises a bioresorbable polymer.

6. The prosthetic device of claim 1, where the porous scaffold comprises a polyolefin, a fluoropolymer, a polysiloxane, a polyurethane, a polyester, a polyimide, a polyetherimide, or a combination thereof.

7. The prosthetic device of claim 1, where the porous scaffold comprises a polypropylene copolymer and a carbohydrate filler.

8. The prosthetic device of claim 1, where the therapeutic cells are present in an amount of about 10,000 cells to about 15,000 cells per square millimeter of surface area of the porous scaffold.

9. The prosthetic device of claim 1, where the therapeutic cells are disposed on the porous scaffold by seeding them into the porous scaffold.

10. A method of repairing a damaged organ in a living being, the method comprising:
modeling the damaged organ from images taken from the living being;
receiving a prosthetic device configured for repairing the damaged organ, the prosthetic device comprising: a porous scaffold adapted for hosting a population of therapeutic cells, the porous scaffold comprising an outer surface and an inner surface separated by a thickness; and wherein a median pore size in the porous scaffold is less than a limiting median dimension for the population of therapeutic cells;
seeding the prosthetic device with therapeutic cells; and
implanting the prosthetic device in the living being.

11. A method of fabricating a prosthetic device adapted for repairing a damaged organ in a living being, the method comprising:
receiving a model of the damaged organ, the model developed from images taken from the living being;
fabricating the prosthetic device by building a porous scaffold adapted for hosting a population of therapeutic cells, the porous scaffold comprising an outer surface and an inner surface separated by a thickness; and wherein a median pore size in the porous scaffold is less than a limiting median dimension for the population of therapeutic cells.

12. The method of claim 11, where the fabricating comprises additive manufacturing of the porous scaffold by fused deposition modeling where a fused deposition model includes instructions for depositing material layer-by-layer to create a three-dimensional object.

13. The method of claim 11, further comprising seeding the porous scaffold with therapeutic cells by bioprinting.

14. The method of claim 11, further comprising seeding the porous scaffold with therapeutic cells while rotating the porous scaffold.

15. The method of claim 11, where the images are collected using computed tomography, magnetic resonance imaging, positron emission tomography, single-photon emission computerized tomography, ultrasound or a combination thereof.

16. The method of claim 15, further comprising:
inducing a paracrine effect in the autologous cells, the paracrine effect facilitating new tissue growth across a span of the scaffold; where the new tissue develops an original tissue of the organ.

17. The method of claim 16, where the autologous cells are stem cells.

18. The method of claim 17, where the stem cells are esophageal epithelial cells, adipose derived mesenchymal stromal cells, muscle cells, neural progenitor cells, induced pluripotent stem cells, bone marrow stem cells or a combination thereof.

19. A prosthetic device for repairing a damaged organ, the prosthetic device comprising:
a porous scaffold adapted for hosting a population of therapeutic cells, the porous scaffold comprising an outer surface and an inner surface separated by a thickness; and
wherein a median pore size in the porous scaffold is less than a limiting median dimension for the population of therapeutic cells; and,
a population of therapeutic cells seeded upon the scaffold, the therapeutic cells comprising autologous cells in an amount of between about 10,000 cells to about 15,000 cells per square millimeter of surface area of the porous scaffold.

20. The prosthetic device as in claim 19, wherein the damaged organ comprises an esophagus.
